(19) United States
(12) Patent Application Publication
(10) Pub. No.: US 2017/0121237 A1
(43) Pub. Date: May 4, 2017

(54) METHODS AND APPARATUS FOR CONVERTING OXYGENATE-CONTAINING FEEDSTOCKS TO GASOLINE AND DISTILLATES

(71) Applicant: ExxonMobil Research and Engineering Company, Annandale, NJ (US)

(72) Inventors: Samia Ilias, Somerville, NJ (US); Brett T. Loveless, Houston, TX (US); Stephen J. McCarthy, Center Valley, PA (US); Brandon O'Neill, Lebanon, NJ (US)

(21) Appl. No.: 15/333,327

(22) Filed: Oct. 25, 2016

Related U.S. Application Data
(60) Provisional application No. 62/247,299, filed on Oct. 28, 2015.

(51) Int. Cl. C07C 1/20; B01J 8/04; C07C 2/12

(52) U.S. Cl. CPC C07C 1/20; C07C 2/12; B01J 8/04; C07C 2529/40; C07C 2529/70

(57) ABSTRACT

Processes for forming refined hydrocarbons are disclosed. Exemplary processes include providing a first mixture comprising ≧10 wt % of at least one oxygenate; contacting at least a portion of the first mixture with a methanol conversion catalyst under suitable conditions including a first pressure, P₁, to yield an intermediate composition including olefins having at least two carbon atoms; introducing at least a portion of the intermediate composition to an oligomerization catalyst under suitable conditions including a second pressure, P₂, to yield an effluent mixture comprising gasoline boiling range components and distillate boiling range components; and recovering at least a portion of the gasoline boiling range components and distillate boiling range components. The first and second pressure can be relatively similar. Apparatus and systems for carrying out the disclosed processes are also described.

### CROSS REFERENCE TO RELATED APPLICATIONS

This application claims the benefit of U.S. Provisional Application No. 62/247,299, filed on Oct. 28, 2015, the entire contents of which are incorporated herein by reference.

### FIELD

The invention is directed to methods and apparatus for converting oxygenate containing feedstocks to gasoline and distillates

### BACKGROUND

In order to provide an adequate supply of synthetic fuels and/or chemical feedstocks, various processes have been developed for converting oxygenated feedstocks, especially methanol, to liquid hydrocarbons. While such processes are known at commercial scale, the demand for heavier hydrocarbons has led to the development of processes for increasing the yield of desirable fuel components by multi-stage techniques.

In a first stage, the oxygenate is converted to a product that includes olefins. The olefins may then be provided to a second stage, in which the olefins are converted to gasoline and distillate fractions. Typically, feedstocks comprising lower olefins, especially C₂-C₅ alkenes are utilized.

Conversion of lower olefins, especially propene and butenes, is effective at moderately elevated temperatures and pressures. The conversion products are sought as liquid fuels, especially the C5⁺ aliphatic and aromatic hydrocarbons. Olefinic gasoline is produced in good yield may be recovered as a product or recycled to the reactor system for further conversion to distillate-range products. Exemplary such processes are described in numerous publications, e.g., U.S. Pat. Nos. 3,960,978; 4,021,502; 4,150,062; 4,211,640; 4,227,992; 4,433,185; 4,445,031; 4,456,779; 4,579,995; 4,929,780; 5,146,032; 5,177,279, and U.S. Published Application Nos. 2011/0152594.

In conventional processes, the catalysts that perform that olefin oligomerization provide acceptable conversion at relatively high pressures relative to those useful in methanol conversion. Thus, the olefin-containing stream produced by the catalyst during methanol conversion is compressed with a compressor to provide acceptable productivity during oligomerization. The compression step is energy intensive and complicates the overall process.

Processes and apparatus that can convert oxygenates to a product from which fuel compositions such as gasoline and distillate fractions may be recovered without the need for compression of the methanol conversion product would be beneficial.

### SUMMARY

Aspects of the invention relate at least in part to the discovery that through careful selection of catalysts, a feed comprising oxygenate, e.g., methanol, dimethylether, mixtures thereof, etc. may be converted to gasoline boiling range components and distillate boiling range components without need for compression between methanol conversion and oligomerization steps.

Thus, in one aspect, embodiments of the invention provide a process for forming a refined hydrocarbon comprising: (a) providing a first mixture comprising ≧10.0 wt % of at least one oxygenate, based on the weight of the first mixture; (b) contacting at least a portion of the feed with a methanol conversion catalyst under suitable conditions including a first pressure, P₁, to yield an intermediate composition including olefins having at least two carbon atoms; (c) introducing at least a portion of the intermediate composition to an oligomerization catalyst under suitable conditions including a second pressure, P₂, to yield an effluent mixture comprising gasoline boiling range components and distillate boiling range components, wherein the P₂=P₁±200 psi, particularly ±175 psi, ±150 psi, ±125 psi, ±100 psi, ±75 psi, ±50 psi, ±40 psi, ±30 psi, ±25 psi, ±20 psi, ±15 psi, ±10 psi, ±5 psi, or ±2.5 psi; and (d) recovering the gasoline boiling range components and distillate boiling range components.

In another aspect embodiments of the invention provide a system for forming a refined hydrocarbon comprising: (a) a feed comprising ≧10 wt % of at least one oxygenate, based on the weight of the first mixture; (b) a first reaction vessel containing a methanol conversion catalyst in fluid communication with at least a portion of the feed for contact with the methanol conversion catalyst maintained under suitable conditions including a first pressure, P₁, to yield an intermediate composition including olefins having at least two carbon atoms; (c) a second reaction vessel containing an oligomerization catalyst in fluid communication with at least a portion of the intermediate composition, the second reaction vessel maintained under suitable conditions including a second pressure, P₂, to yield and effluent mixture comprising gasoline boiling range components and distillate boiling range components; and (d) a recovery system in fluid communication with the second reaction vessel to separate the gasoline boiling range components and distillate boiling range components from the effluent mixture, wherein the P₂=P₁±200 psi, particularly ±175 psi, ±150 psi, ±125 psi, ±100 psi, ±75 psi, ±50 psi, ±40 psi, ±30 psi, ±25 psi, ±20 psi, ±15 psi, ±10 psi, ±5 psi, or ±2.5 psi.

Still another aspect of the invention provides a system for forming a refined hydrocarbon comprising: (a) a feed comprising ≧10 wt % of at least one oxygenate, based on the weight of the first mixture; (b) a first reaction vessel containing a methanol conversion catalyst in fluid communication with at least a portion of the feed for contact with the methanol conversion catalyst maintained under suitable conditions including a first pressure, P₁, to yield an intermediate composition including olefins having at least two carbon atoms, thereafter maintained under a second set of conditions including second pressure P₂, to yield and effluent mixture comprising gasoline boiling range components and distillate boiling range components, wherein the P₂=P₁±200 psi, particularly ±175 psi, ±150 psi, ±125 psi, ±100 psi, ±75 psi, ±50 psi, ±40 psi, ±30 psi, ±25 psi, ±20 psi, ±15 psi, ±10 psi, ±5 psi, or ±2.5 psi; and (c) a recovery system in fluid communication with the second reaction vessel to separate at least a portion of the gasoline boiling range components and distillate boiling range components from the effluent mixture.

### BRIEF DESCRIPTION OF THE DRAWINGS

FIG. 1 schematically illustrates a dual reactor process and apparatus according to embodiments of the invention.

FIG. 2 schematically illustrates a single reactor process and apparatus according to embodiments of the invention.

### DETAILED DESCRIPTION

As used herein, the term “produced in an industrial scale” refers to a production scheme in which gasoline and/or distillate end products are produced on a continuous basis (with the exception of necessary outages for plant maintenance) over an extended period of time (e.g., over at least a week, or a month, or a year) with the expectation of generating revenues from the sale or distribution of the gas and/or distillate. Production at an industrial scale is distinguished from laboratory or pilot plant settings which are typically maintained only for the limited period of the experiment or investigation, and are conducted for research purposes and not with the expectation of generating revenue from the sale or distribution of the gasoline or distillate produced thereby.

As used herein, and unless specified otherwise, “gasoline” or “gasoline boiling range components” refers to a composition containing at least predominantly C₅-C₁₂ hydrocarbons. In one embodiment, gasoline or gasoline boiling range components is further defined to refer to a composition containing at least predominantly C₅-C₁₂ hydrocarbons and further having a boiling range of from about 100° F. to about 400° F. In an alternative embodiment, gasoline or gasoline boiling range components is defined to refer to a composition containing at least predominantly C₅-C₁₂ hydrocarbons, having a boiling range of from about 100° F. to about 400° F., and further defined to meet ASTM standard D439.

As used herein, and unless specified otherwise, the term “distillate” or “distillate boiling range components” refers to a composition containing predominately C₁₀-C₃₀ hydrocarbons. In one embodiment, distillate or distillate boiling range components is further defined to refer to a composition containing at least predominately C₁₀-C₃₀ hydrocarbons and further having a boiling range of from about 300° F. to about 700° F. Examples of distillates or distillate boiling range components include, but are not limited to, naphtha, jet fuel, diesel, kerosene, aviation gas, fuel oil, heating oil and blends thereof.

As used herein, and unless specified otherwise, the term “diesel” refers to middle distillate fuels containing at least predominantly C₁₀-C₂₅ hydrocarbons. In one embodiment, diesel is further defined to refer to a composition containing at least predominantly C₁₀-C₂₅ hydrocarbons, and further having a boiling range of from about 330° F. to about 700° F. In an alternative embodiment, diesel is as defined above to refer to a composition containing at least predominantly C₁₀-C₂₅ hydrocarbons, having a boiling range of from about 330° F. to about 700° F., and further defined to meet ASTM standard D975.

As used herein the phase “essentially free of compression step” means that the intermediate composition is not caused to go through a compressor or provided to a vessel or conduit that causes the pressure to increase ≧2.5 psi.

For the purposes of this invention and the claims thereto, the new numbering scheme for the Periodic Table Groups is used as described in Chemical and Engineering News, 63(5), pg. 27 (1985).

As used herein references to a “reactor,” “reaction vessel,” and the like shall be understood to include both distinct reactors as well as reaction zones within a single reactor apparatus. In other words and as is common, a single reactor may have multiple reaction zones.

Where the description refers to a first and second reactor, the person of ordinary skill in the art will readily recognize such reference includes a single reactor having first and second reaction zones. Likewise, a first reactor effluent and a second reactor effluent will be recognized to include the effluent from the first reaction zone and the second reaction zone of a single reactor, respectively.

As used herein the phrase “at least a portion of” means >0 to 100 wt % of the process stream or composition to which the phrase refers. The phrase “at least a portion of” refers to an amount≦about 1 wt %, ≦about 2 wt %, ≦about 5 wt %, ≦about 10 wt %, ≦about 20 wt %, ≦about 25 wt %, ≦about 30 wt %, ≦about 40 wt %, ≦about 50 wt %, ≦about 60 wt %, ≦about 70 wt %, ≦about 75 wt %, ≦about 80 wt %, ≦about 90 wt %, ≦about 95 wt %, ≦about 98 wt %, ≦about 99 wt %, or ≦about 100 wt %. Additionally or alternatively, the phrase “at least a portion of” refers to an amount ≧about 1 wt %, ≧about 2 wt %, ≧about 5 wt %, ≧about 10 wt %, ≧about 20 wt %, ≧about 25 wt %, ≧about 30 wt %, ≧about 40 wt %, ≧about 50 wt %, ≧about 60 wt %, ≧about 70 wt %, ≧about 75 wt %, ≧about 80 wt %, ≧about 90 wt %, ≧about 95 wt %, ≧about 98 wt %, or ≧about 99 wt %. Ranges expressly disclosed include all combinations of any of the above-enumerated values; e.g., ˜10 wt % to ˜100 wt %, ˜10 wt % to ˜98 wt %, ˜2 wt % to ˜10 wt %, ˜40 wt to ˜60 wt %, etc.

As used herein the term “first mixture” means a hydrocarbon-containing composition including one or more oxygenates. Typically, the first mixture comprises ≧10 wt % of at least one oxygenate, based on the weight of the first mixture. Thus, the amount of oxygenate(s) in the first mixture may be ≧10 wt %, ≧about 12.5 wt %, ≧about 15 wt %, ≧about 20 wt %, ≧about 25 wt %, ≧about 30 wt %, ≧about 35 wt %, ≧about 40 wt %, ≧about 45 wt %, ≧about 50 wt %, ≧about 55 wt %, ≧about 60 wt %, ≧about 65 wt %, ≧about 70 wt %, ≧about 75 wt %, ≧about 80 wt %, ≧about 85 wt %, ≧about 90 wt %, ≧about 95 wt %, ≧about 99 wt %, ≧about 99.5 wt %, or about 100 wt %. Additionally or alternatively, the amount of oxygenate in the first mixture may be ≦about 12.5 wt %, ≦about 15 wt %, ≦about 20 wt %, ≦about 25 wt %, ≦about 30 wt %, ≦about 35 wt %, ≦about 40 wt %, ≦about 45 wt %, ≦about 50 wt %, ≦about 55 wt %, ≦about 60 wt %, ≦about 65 wt %, ≦about 70 wt %, ≦about 75 wt %, ≦about 80 wt %, ≦about 85 wt %, ≦about 90 wt %, ≦about 95 wt %, ≦about 99 wt %, ≦about 99.5 wt %, or ≦about 100 wt %. Ranges expressly disclosed include all combinations of any of the above-enumerated values; e.g., ≧10 wt % to about 100 wt %, about 12.5 wt % to about 99.5 wt %, about 20 wt % to about 90 wt %, about 50 wt % to about 99 wt %, etc.

As used herein the term “oxygenate,” “oxygenate composition,” and the like refer to oxygen-containing compounds and mixtures of oxygen-containing compounds that have 1 to about 50 carbon atoms, 1 to about 20 carbon atoms, 1 to about 10 carbon atoms, or 1 to 4 carbon atoms. Exemplary oxygenates include alcohols, ethers, carbonyl compounds, e.g., aldehydes, ketones and carboxylic acids, and mixtures thereof. Particular oxygenates methanol, ethanol, dimethyl ether, diethyl ether, methylethyl ether, di-isopropyl ether, dimethyl carbonate, dimethyl ketone, formaldehyde, and acetic acid.

In any aspect, the oxygenate comprises one or more alcohols, preferably alcohols having 1 to about 20 carbon atoms, 1 to about 10 carbon atoms, or 1 to 4 carbon atoms. The alcohols useful as first mixtures may be linear or branched, substituted or unsubstituted aliphatic alcohols and their unsaturated counterparts. Non-limiting examples of such alcohols include methanol, ethanol, propanols (e.g., n-propanol, isopropanol), butanols (e.g., n-butanol, sec-butanol, tert-butyl alcohol), pentanols, hexanols, etc., and mixtures thereof. In any aspect described herein, the first mixture may be one or more of methanol, and/or ethanol, particularly methanol. In any aspect, the first mixture may be methanol and dimethyl ether.

The oxygenate, particularly where the oxygenate comprises an alcohol (e.g., methanol), may optionally be subjected to dehydration, e.g., catalytic dehydration over e.g., γ-alumina. Further optionally, at least a portion of any methanol and/or water remaining in the first mixture after catalytic dehydration may be separated from the first mixture. If desired, such catalytic dehydration may be used to reduce the water content of reactor effluent before it enters a subsequent reactor or reaction zone, e.g., second and/or third reactors as discussed below.

In any aspect, one or more other compounds may be present in first mixture. Some common or useful such compounds have 1 to about 50 carbon atoms, 1 to about 20 carbon atoms, 1 to about 10 carbon atoms, or 1 to 4 carbon atoms. Typically, although not necessarily, such compounds include one or more heteroatoms other than oxygen. Some such compounds include amines, halides, mercaptans, sulfides, and the like. Particular such compounds include alkyl-mercaptans (e.g., methyl mercaptan and ethyl mercaptan), alkyl-sulfides (e.g., methyl sulfide), alkyl-amines (e.g., methyl amine), and alkyl-halides (e.g., methyl chloride and ethyl chloride). In any aspect, the first mixture includes one or more of 1.0 wt % acetylene, pyrolysis oil or aromatics, particularly C6 and/or C7 aromatics. Thus, the amount of such other compounds in the first mixture may be ≦about 2.0 wt %, ≦about 5.0 wt %, ≦about 10 wt %, ≦about 15 wt %, ≦about 20 wt %, ≦about 25 wt %, ≦about 30 wt %, ≦about 35 wt %, ≦about 40 wt %, ≦about 45 wt %, ≦about 50 wt %, ≦about 60 wt %, ≦about 75 wt %, ≦about 90 wt %, or ≦about 95 wt %. Additionally or alternatively, the amount of such other compounds in the first mixture may be ≧about 2.0 wt. %, ≧about 5.0 wt %, ≧about 10 wt %, ≧about 15 wt %, ≧about 20 wt %, ≧about 25 wt %, ≧about 30 wt %, ≧about 35 wt %, ≧about 40 wt %, ≧about 45 wt %, ≧about 50 wt %, ≧about 60 wt %, ≧about 75 wt %, or ≧about 90 wt %. Ranges expressly disclosed include all combinations of any of the above-enumerated values; e.g., 1.0 wt % to about 10.0 wt %, about 2.0 wt % to about 5.0 wt %, about 10 wt % to about 95 wt %, about 15 wt % to about 90 wt %, about 20 wt % to about 75 wt %, about 25 wt % to about 60 wt %, about 30 wt % to about 50 wt %, about 35 wt % to about 45 wt %, etc.

Reference will now be made to various aspects and embodiments of the disclosed subject matter in view of the definitions above. Reference to the systems will be made in conjunction with, and understood from, the method disclosed herein.

### Methanol to Olefin Reaction Conditions

As noted above, embodiments of the presently disclosed subject matter include a stage in which a feed comprising an oxygenate, e.g., methanol, dimethyl ether, or a mixture thereof is introduced to a reaction vessel having a methanol conversion catalyst therein. The reaction vessel is controlled to provide conditions suitable for the catalyst to convert at least a portion of the oxygenate to an intermediate composition comprising one or more olefins having 2 or more carbon atoms, sometimes referred to as a light C2+ olefin composition. This process is known as a MTO (methanol to olefin) reaction.

Embodiments of the invention include contacting at least a portion of the feed with a methanol conversion catalyst under suitable conditions including a first pressure, P₁, to yield an intermediate composition including olefins having at least two carbon atoms. In any embodiment, the pressure of the reaction vessel during methanol conversion may be ≧about 5.0 psig, e.g., ≧about 10 psig, ≧about 25 psig, ≧about 50 psig, ≧about 75 psig, ≧about 100 psig, ≧about 125 psig, ≧about 150 psig, ≧about 200 psig, ≧about 250 psig, ≧about 300 psig, ≧about 350 psig, ≧about 400 psig, or ≧about 450 psig. Additionally or alternatively, the pressure of the reaction vessel during methanol conversion may be ≦about 500 psig, e.g., ≦about 450 psig, ≦about 400 psig, ≦about 350 psig, ≦about 300 psig, ≦about 250 psig, ≦about 200 psig, ≦about 150 psig, ≦about 125 psig, ≦about 100 psig, ≦about 75 psig, ≦about 50 psig, ≦about 25 psig, or ≦about 10 psig. Ranges of the pressure of reaction during methanol conversion expressly disclosed include all combinations of any of the above-enumerated values; e.g., about 5.0 psig to about 500 psig, about 10 psig to about 450 psig, about 25 psig to about 400 psig, about 50 psig to about 350 psig, about 75 psig to about 300 psig, about 100 psig to about 250 psig, about 125 psig to about 200 psig, about 25 psig to about 75 psig, about 50 psig to about 125 psig, about 75 psig to about 100 psig, about 85 to about 95 psig. etc.

The temperature of reaction during methanol conversion may be from about ≧about 250° C., e.g., ≧about 275° C., ≧about 300° C., ≧about 330° C., ≧about 350° C., ≧about 375° C., ≧about 400° C., ≧about 425° C., to about 450° C., ≧about 500° C., ≧about 525° C., ≧about 550° C., or ≧about 575° C. Additionally or alternatively, the temperature of reaction during methanol conversion may be ≦about 600° C., e.g., ≦about 575° C., ≦about 550° C., ≦about 525° C., ≦about 500° C., ≦about 450° C., ≦about 425° C., ≦about 400° C., ≦about 375° C., ≦about 350° C., ≦about 330° C., ≦about 300° C., or ≦about 275° C. Ranges of the temperature of reaction during methanol conversion expressly disclosed include all combinations of any of the above-enumerated values; e.g., about 250° C. to about 600° C., about 275° C. to about 575° C., about 330° C. to about 550° C., about 350° C. to about 525° C., about 375° C. to about 500° C., about 400° C. to about 475° C., about 425° C. to about 450° C., about 400° C. to about 500° C., about 425° C. to about 500° C., about 450° C. to about 500° C., about 475° C. to about 500° C., etc.

The weight hourly space velocity (WHSV) of feed stock during methanol conversion may be ≧about 0.1 hr⁻¹, e.g., ≧about 1.0 hr⁻¹, ≧about 10 hr⁻¹, ≧about 50 hr⁻¹, ≧about 100 hr⁻¹, ≧about 200 hr⁻¹, ≧about 300 hr⁻¹, or ≧about 400 hr⁻¹. Additionally or alternatively, the WHSV may be ≦about 500 hr⁻¹, e.g., ≦about 400 hr⁻¹, ≦about 300 hr⁻¹, ≦about 200 hr⁻¹, ≦about 100 hr⁻¹, ≦about 50 hr⁻¹, ≦about 10 hr⁻¹, or ≦about 1.0 hr⁻¹. Ranges of the WHSV expressly disclosed include all combinations of any of the above-enumerated values; e.g., from about 0.1 hr⁻¹ to about 500 hr⁻¹, from about 0.5 hr⁻¹ to about 100 hr⁻¹, from about 1.0 hr⁻¹ to about 10 hr⁻¹, from about 2.0 hr⁻¹ to about 5.0 hr⁻¹, etc.

In any embodiment, combinations of the above described ranges of the WHSV, temperature and pressures may be employed for the methanol conversion. For example in some embodiments, the temperature of the reaction vessel during methanol conversion may be from about 400° C. to about 600° C., e.g., about 425° C. to about 550° C., about 450° C. to about 500° C., about 475° C. to about 500° C., or at about 485° C.; the WHSV may be about 0.1 hr⁻¹ to about 10 hr⁻¹, e.g., about 0.5 hr⁻¹ to about 8.0 hr⁻¹, about 0.75 hr⁻¹ to about 5.0 hr⁻¹, about 1.0 hr⁻¹ to about 4.0 hr⁻¹, or about 2.0 hr⁻¹ to about 3.0 hr⁻¹; and/or the pressure may be about 50 psig to about 200 psig, e.g., about 75 psig to about 150 psig or about 75 psig to about 100 psig. All combinations and permutations of these values are expressly disclosed. For example, in particular embodiments, the temperature may be about 475° C. to about 500° C., the WHSV may be about 1.0 hr⁻¹ to about 4.0 hr⁻¹, and the pressure may be 75 psig to about 100 psig.

The methanol conversion catalyst may be selected from aluminosilicate zeolites and silicoaluminophosphate zeotype materials. Typically, such materials useful herein have a microporous surface area ≧150 m²/g, e.g., ≧155 m²/g, 160 m²/g, 165 m²/g, ≧200 m²/g, ≧250 m²/g, ≧300 m²/g, ≧350 m²/g, ≧400 m²/g, ≧450 m²/g, ≧500 m²/g, ≧550 m²/g, ≧600 m²/g, ≧650 m²/g, ≧700 m²/g, ≧750 m²/g, ≧800 m²/g, ≧850 m²/g, ≧900 m²/g, ≧950 m²/g, or ≧1000 m²/g. Additionally or alternatively, the surface area may be ≦1200 m²/g, e.g., ≦1000 m²/g, ≦950 m²/g, ≦900 m²/g, ≦850 m²/g, ≦800 m²/g, ≦750 m²/g, ≦700 m²/g, ≦650 m²/g, ≦600 m²/g, ≦550 m²/g, ≦500 m²/g, ≦450 m²/g, ≦400 m²/g, ≦350 m²/g, ≦250 m²/g, ≦200 m²/g, ≦165 m²/g, ≦160 m²/g, or ≦155 m²/g. Ranges of the surface area expressly disclosed include all combinations of any of the above-enumerated values; e.g., 150 m²/g to 1200 m²/g, 160 m²/g to about 1000 m²/g, 165 m²/g to 950 m²/g, 200 m²/g to 900 m²/g, 250 m²/g to 850 m²/g, 300 m²/g to 800 m²/g, 275 m²/g to 750 m²/g, 300 m²/g to 700 m²/g, 350 m²/g to 650 m²/g, 400 m²/g to 600 m²/g, 450 m²/g to 550 m²/g, etc.

The methanol conversion catalyst may have any ratio of silicon to aluminum. Particular catalysts have a molar ratio of silicon to aluminum ≧about 10, e.g., ≧about 20, ≧about 30, ≧about 40, ≧about 42, ≧about 45, ≧about 48, ≧about 50, ≧about 60, ≧about 70, ≧about 80, or ≧about 90. Additionally or alternatively, the methanol conversion catalyst may have a molar ratio of silicon to aluminum ≦about 100, e.g., ≦about 90, ≦about 80, ≦about 70, ≦about 60, ≦about 50, ≦about 48, ≦about 45, ≦about 42, ≦about 40, ≦about 30, or ≦about 20. Ranges of the molar ratio expressly disclosed include all combinations of any of the above-enumerated values; e.g., about 10 to about 100, about 20 to about 90, about 30 to about 80, about 40 to about 70, about 40 to about 60, about 45 to about 50, about 30 to about 50, about 42 to about 48, etc. The silicon: aluminum ratio may be selected or adjusted to provide a desired activity and/or a desired distribution of molecules from the methanol conversion.

Additionally or alternatively, particular aluminosilicate zeolites useful as methanol conversion catalysts have a hexane cracking activity (also referred to as “alpha-activity” or as “alpha value”) ≧about 20, e.g., ≧about 40, ≧about 60, ≧about 80, ≧about 100, ≧about 120, >about 140, ≧about 160, or ≧about 180. Additionally or alternatively, the hexane cracking activity of the methanol conversion catalyst may be ≦about 200, e.g., ≦about 180, ≦about 160, ≦about 140, ≦about 120, ≦about 100, ≦about 80, ≦about 60, ≦about 40. Ranges of the alpha values expressly disclosed include all combinations of any of the above-enumerated values; e.g., ˜20 to ˜200, ˜40 to ˜180, ˜60 to ˜160, ˜80 to ˜140, ˜100 to ˜120, etc. Hexane cracking activity according to the alpha test is described in U.S. Pat. No. 3,354,078; in the Journal of Catalysis at vol. 4, p. 527 (1965), vol. 6, p. 278 (1966), and vol. 61, p. 395 (1980), each incorporated herein by reference as to that description. The experimental conditions of the test used herein include a constant temperature of about 538° C. and a variable flow rate as described in detail in the Journal of Catalysis at vol. 61, p. 395. Higher alpha values typically correspond to a more active cracking catalyst.

Aluminosilicate zeolites useful as methanol conversion catalyst may be characterized by an International Zeolite Associate (IZA) Structure Commission framework type selected from the group consisting of BEA, EUO, FER, IMF, LAU, MEL, MFI, MRE, MFS, MTT, MWW, NES, TON, SFG, STF, STI, TUN, PUN, and combinations and intergrowths thereof.

Particular examples of suitable methanol conversion catalysts can include ZSM-5, ZSM-11, ZSM-12, ZSM-22, ZSM-23, ZSM-35, and ZSM-48 as well as combinations thereof. Particularly useful catalysts can include zeolites having an MRE-type IZA framework, e.g., ZSM-48 catalyst, particularly where improved conversion to distillate is desired. Other particularly useful catalysts may include zeolites having an MFI-type IZA framework, e.g., H-ZSM-5 catalyst, particularly for distillate feeds, provided the catalyst has been steamed as is known in the art. In some embodiments, the catalyst may include or be ZSM-12. Catalyst activity may be modified, e.g., by use of catalysts that are not fully exchanged. Activity is also known to be affected by the silicon: aluminum ratio of the catalyst. For example, catalysts prepared to have a higher silica: aluminum ratio can tend to have lower activity. The person of ordinary skill will recognize that the activity can be modified to give the desired low aromatic product in methanol conversion.

Zeolite ZSM-5 and the conventional preparation thereof are described in U.S. Pat. No. 3,702,886. Zeolite ZSM-11 and the conventional preparation thereof are described in U.S. Pat. No. 3,709,979. Zeolite ZSM-12 and the conventional preparation thereof are described in U.S. Pat. No. 3,832,449. Zeolite ZSM-23 and the conventional preparation thereof are described U.S. Pat. No. 4,076,842. Zeolite ZSM-35 and the conventional preparation thereof are described in U.S. Pat. No. 4,016,245. ZSM-48 and the conventional preparation thereof are taught by U.S. Pat. No. 4,375,573. The entire disclosures of these U.S. patents are incorporated herein by reference.

Exemplary silicoaluminophosphates that may be useful herein can include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, and SAPO-56.

The selectivity of these catalysts for may be modified as is known in the art to provide for little or no aromatics formation, particularly where improved distillate formation is desired, such that intermediate composition exiting the methanol conversion reactor comprises ≧about 80 wt % olefin, e.g., ≧about 82.5 wt % olefin, ≧about 85 wt % olefin, ≧about 87.5 wt % olefin, ≧about 90 wt % olefin, ≧about 92.5 wt % olefin, ≧about 95 wt % olefin, ≧about 97.5 wt % olefin, ≧about 99 wt % olefin, or ≧about 99.5 wt % olefin. Additionally or alternatively, effluent exiting the methanol conversion reactor may comprise ≦about 100 wt % olefin, e.g., ≦about 99.5 wt % olefin, ≦about 99 wt % olefin, ≦about 97.5 wt % olefin, ≦about 95 wt % olefin, ≦about 92.5 wt % olefin, ≦about 90 wt % olefin, ≦about 87.5 wt % olefin, ≦about 85 wt % olefin, or ≦about 82.5 wt % olefin. Ranges of the amount of olefin in the intermediate composition include all combinations of any of the above-enumerated values; e.g., about 80 wt % to about 100 wt % olefin, about 82.5 wt % to about 99.5 wt % olefin, about 85 wt % to about 99 wt % olefin, about 87.5 wt % to about 97.5 wt % olefin, about 90 wt % to about 95 wt % olefin, etc.

In certain embodiments, e.g., where gasoline boiling range components are more desired, the catalyst may be selected and/or treated to provide an intermediate composition comprising lesser amounts of olefin. Typically, in such embodiments it is desirable that the intermediate composition comprise ≧about 30 wt %, e.g., ≧about 35 wt %, ≧about 40 wt %, about 45 wt %, ≧about 50 wt %, ≧about 55 wt %, ≧about 60 wt %, ≧about 65 wt %, ≧about 70 wt %, or ≧about 75 wt % olefins. Ranges of the amount of olefin in the intermediate composition include all combinations of any of the above-enumerated values; e.g., about 30 wt % to about 80 wt. %, about 35 wt % to about 75 wt %, about 40 wt % to about 70 wt %, about 45 wt % to about 65 wt %, about 50 wt % to about 60 wt %, etc.

Thus, the relative amount of aromatic compounds produced by the catalyst may be selected according to the desired composition of the intermediate stream. The aromatics content may be ≦about 50 wt %, e.g., ≦about 45 wt %, ≦about 40 wt %, ≦about 35 wt %, ≦about 30 wt %, ≦about 25 wt %, ≦about 20 wt %, ≦about 15 wt %, ≦about 10 wt %, ≦about 5.0 wt %, ≦about 2.5 wt %, ≦about 1.0 wt %. Additionally or alternatively, the aromatics content of the stream exiting the methanol conversion reactor may be ≧about 1.0 wt %, e.g., ≧about 2.5 wt %, ≧about 5.0 wt %, ≧about 10 wt %, ≧about 15 wt %, ≧about 20 wt %, ≧about 25 wt %, ≧about 30 wt %, ≧about 35 wt %, ≧about 40 wt %, or ≧about 45 wt %.

### Olefin to Gasoline/Distillate Reaction Conditions

Embodiments of the invention include introducing at least a portion of the intermediate composition produced during methanol conversion to an oligomerization catalyst under suitable conditions including a second pressure, P₂, to yield an effluent mixture comprising gasoline boiling range components and distillate boiling range components. The second pressure, P₂, may be selected from values and ranges enumerated above for P₁. Typically, however, P₂ can be selected to be relatively similar to P₁, e.g., P₂=P₁±200 psig, particularly P₂=P₁±175 psig, P₂=P₁±150 psig, P₂=P₁±125 psig, P₂=P₁±100 psig, P₂=P₁±75 psig, P₂=P₁±50 psig, P₂=P₁±40 psig, P₂=P₁±30 psig, P₂=P₁±25 psig, P₂=P₁±20 psig, P₂=P₁±15 psig, P₂=P₁±10 psig, P₂=P₁±5 psig, or P₂=P₁±2.5 psig. Selection of a second pressure, P₂, during oligomerization relatively similar to P₁ reduces or eliminates the cost and energy for compression of the intermediate composition before its introduction to the oligomerization catalyst. Thus, in some embodiments, P₂ and P₁ can be essentially equal, e.g., P₂=P₁±2.0 psig, P₂=P₁±1.0 psig, P₂=P₁±0.5 psig, or P₂=P₁. Thus, embodiments may be essentially free of a compression step/compressor that compresses the intermediate composition before its introduction to the oligomerization catalyst. In some embodiments, the intermediate composition is not intentionally subject to a compressor and/or to compression before its introduction to the oligomerization catalyst.

In embodiments where the oligomerization reaction occurs in a second reaction vessel, the weight hourly space velocity (WHSV) of feed stock during methanol conversion may be ≧about 0.1 hr⁻¹, e.g., ≧about 1.0 hr⁻¹, ≧about 10 hr⁻¹, ≧about 50 hr⁻¹, ≧about 100 hr⁻¹, ≧about 200 hr⁻¹, ≧about 300 hr⁻¹, or ≧about 400 hr⁻¹. Additionally or alternatively, the WHSV may be ≦about 500 hr⁻¹, e.g., ≦about 400 hr⁻¹, ≦about 300 hr⁻¹, ≦about 200 hr⁻¹, ≦about 100 hr⁻¹, ≦about 50 hr⁻¹, ≦about 10 hr⁻¹, or ≦about 1.0 hr⁻¹. Ranges of the WHSV expressly disclosed include all combinations of any of the above-enumerated values; e.g., from about 0.1 hr⁻¹ to about 500 hr⁻¹, from about 0.5 hr⁻¹ to about 100 hr⁻¹, from about 1.0 hr⁻¹ to about 10 hr⁻¹, from about 2.0 hr⁻¹ to about 5.0 hr⁻¹, etc.

The temperature during oligomerization can typically be ≧about 100° C., e.g., ≧about 125° C., ≧about 150° C., ≧about 175° C., ≧about 200° C., ≧about 225° C., ≧about 250° C., or ≧about 275° C. Additionally or alternatively, the temperature during oligomerization may be ≦about 300° C., e.g., ≦about 275° C., ≦about 250° C., ≦about 225° C., ≦about 200° C., ≦about 175° C., ≦about 150° C., or ≦about 125° C. Ranges of the temperature during oligomerization of the intermediate composition expressly disclosed include all combinations of any of the above-enumerated values; e.g., about 100° C. to about 300°, about 125° to about 270° C., about 150° C. to about 250° C., about 175° C. to about 225° C., etc.

In any embodiment, combinations of the above described ranges of the WHSV, temperatures, and pressures may be employed for the oligomerization of the intermediate composition. For example in some embodiments, the temperature of the reaction vessel during oligomerization may be from about 100° C. to about 300° C., e.g., about 150° C. to about 250° C., about 175° C. to about 225° C., etc; the WHSV may be about 0.1 hr⁻¹ to about 10 hr⁻¹, e.g., 0.5 hr⁻¹ to about 8.0 hr⁻¹, 0.75 hr⁻¹ to about 5.0 hr⁻¹, about 1.0 hr⁻¹ to about 4.0 hr⁻¹, or about 2.0 hr⁻¹ to about 3.0 hr⁻¹, etc.; and/or the second pressure, P₂, may be about 50 psig to about 200 psig, e.g., about 75 psig to about 150 psig or about 75 psig to about 100 psig, with the proviso that it is within an above described range of the first pressure, P₁. All combinations and permutations of these values are expressly disclosed. For example, in particular embodiments, the temperature may be about 175° C. to about 225° C., the WHSV may be about 1.0 hr⁻¹ to about 4.0 hr⁻¹, and the pressure may be 75 psig to about 100 psig.

The oligomerization produces an effluent mixture comprising an effluent mixture comprising gasoline boiling range components and distillate boiling range components. Typically the alkylation effluent comprises ≧about 20 wt % of gasoline boiling range components and distillate boiling range components, based on the weight the effluent mixture. In any aspect, the amount of gasoline boiling range components and distillate boiling range components in the effluent mixture may be about 25 wt % to about 100 wt %, about 35 wt % to about 100 wt %, about 50 wt % to about 100 wt %, about 60 wt % to about 100 wt %, about 70 wt % to about 100 wt %, about 80 wt % to about 100 wt %, about 90 wt % to about 100 wt %, about 95 wt % to about 100 wt %; about 30 wt % to about 95 wt %, about 40 wt % to about 95 wt %, about 50 wt % to about 95 wt %, about 60 wt % to about 95 wt %, about 70 wt % to about 95 wt %, about 80 wt % to about 95 wt %, about 90 wt % to about 95 wt %, about 30 wt % to about 90 wt %, about 40 wt % to about 90 wt %, about 50 wt % to about 90 wt %, about 60 wt % to about 90 wt %, about 70 wt % to about 90 wt %, about 80 wt % to about 90 wt %, about 30 wt % to about 80 wt %, about 40 wt % to about 80 wt %, about 50 wt % to about 80 wt %, about 60 to about 80 wt %, about 70 wt % to about 80 wt %, about 30 wt % to about 70.0 wt %, about 40 wt % to about 70 wt %, about 50 wt % to about 70 wt %, about 60.0 to about 70 wt %, about 30 wt % to about 60 wt %, about 40.0 wt % to about 60 wt %, about 25 wt % to about 50 wt %, about 30 wt % to about 40 wt %, about 30 wt % to about 50 wt %, about 40 wt % to about 50 wt %, etc.

In particular embodiments, the effluent mixture may comprise ≧about 50 wt %, e.g., ≧about 55 wt %, ≧about 60 wt %, ≧about 65 wt %, ≧about 70 wt %, ≧about 75 wt %, ≧about 80 wt %, ≧about 85 wt %, ≧about 90 wt %, ≧about 95 wt %, or ≧about 99 wt % distillate boiling range components, based on the weight the effluent mixture. Additionally or alternatively, the effluent mixture may comprise ≦about 100 wt %, e.g., ≦about 99 wt %, ≦about 95 wt %, ≦about 90 wt %, ≦about 85 wt %, ≦about 80 wt %, ≦about 75 wt %, ≦about 70 wt %, ≦about 65 wt %, ≦about 60 wt %, or ≦about 55 wt %. Ranges of the amount of distillate boiling range components in the effluent mixture expressly disclosed include all combinations of any of the above-enumerated values, e.g., about 50 wt % to about 99 wt %, about 55 wt % to about 95 wt %, about 60 wt % to about 90 wt %, about 65 wt % to about 85 wt %, etc.

The oligomerization catalyst may be selected from aluminosilicate zeolites and silicoaluminophosphate zeotype materials. Typically, such materials useful herein can have a microporous surface area ≧150 m²/g, e.g., ≧155 m²/g, 160 m²/g, 165 m²/g, ≧200 m²/g, ≧250 m²/g, ≧300 m²/g, ≧350 m²/g, ≧400 m²/g, ≧450 m²/g, ≧500 m²/g, ≧550 m²/g, ≧600 m²/g, ≧650 m²/g, ≧700 m²/g, ≧750 m²/g, ≧800 m²/g, ≧850 m²/g, ≧900 m²/g, ≧950 m²/g, or ≧1000 m²/g. Additionally or alternatively, the surface area may be ≦1200 m²/g, e.g., ≦1000 m²/g, ≦950 m²/g, ≦900 m²/g, ≦850 m²/g, ≦800 m²/g, ≦750 m²/g, ≦700 m²/g, ≦650 m²/g, ≦600 m²/g, ≦550 m²/g, ≦500 m²/g, ≦450 m²/g, ≦400 m²/g, ≦350 m²/g, ≦250 m²/g, ≦200 m²/g, ≦165 m²/g, ≦160 m²/g, or ≦155 m²/g. Ranges of the surface area expressly disclosed include all combinations of any of the above-enumerated values; e.g., 150 m²/g to 1200 m²/g, 160 m²/g to about 1000 m²/g, 165 m²/g to 950 m²/g, 200 m²/g to 900 m²/g, 250 m²/g to 850 m²/g, 300 m²/g to 800 m²/g, 275 m²/g to 750 m²/g, 300 m²/g to 700 m²/g, 350 m²/g to 650 m²/g, 400 m²/g to 600 m²/g, 450 m²/g to 550 m²/g, etc.

The oligomerization catalyst may have any ratio of silicon to aluminum. Particular oligomerization catalysts have a molar ratio of silicon to aluminum ≧about 10, e.g., ≧about 20, ≧about 30, ≧about 40, ≧about 42, ≧about 45, ≧about 48, ≧about 50, ≧about 60, ≧about 70, ≧about 80, or ≧about 90. Additionally or alternatively, the oligomerization catalyst may have a molar ratio of silicon to aluminum ≦about 100, e.g., ≦about 90, ≦about 80, ≦about 70, ≦about 60, ≦about 50, ≦about 48, ≦about 45, ≦about 42, ≦about 40, ≦about 30, or ≦about 20. Ranges of the surface area expressly disclosed include all combinations of any of the above-enumerated values; e.g., about 10 to about 100, about 20 to about 90, about 30 to about 80, about 40 to about 70 about 42 to about 60, about 45 to about 50, about 30 to about 50, about 42 to about 48.

Additionally or alternatively, particular aluminosilicate zeolites and silicoaluminophosphate zeotype materials useful as oligomerization catalysts have an alpha activity ≧about 20, e.g., ≧about 40, ≧about 60, ≧about 80, ≧about 100, ≧about 120, ≧about 140, ≧about 160, or ≧about 180. Additionally or alternatively, the alpha activity of the oligomerization catalyst may be ≦about 200, e.g., ≦about 180, ≦about 160, ≦about 140, ≦about 120, ≦about 100, ≦about 80, ≦about 60, ≦about 40. Ranges of the surface area expressly disclosed include all combinations of any of the above-enumerated values; e.g., about 20 to about 200, about 40 to about 180, about 60 to about 160, about 80 to about 140, about 100 to about 120, etc.

As disclosed in U.S. Pat. No. 7,361,798, which is hereby incorporated in its entirety by reference herein, zeolites are classified by the Structure Commission of the International Zeolite Association (IZA) according to the rules of the IUPAC Commission on Zeolite Nomenclature. A framework-type describes the topology and connectivity of the tetrahedrally coordinated atoms constituting the framework and makes an abstraction of the specific properties for those materials. Molecular sieves for which a structure has been established are assigned a three letter code and are described in the Atlas of Zeolite Framework Types, 5^{th} edition, Elsevier, London, England (2001), which is incorporated in its entirety by reference herein. Aluminosilicate zeolites useful as oligomerization catalyst may optionally be characterized by an International Zeolite Associate (IZA) Structure Commission framework comprising BEA, EUO, FER, IMF, LAU, MEL, MFI, MRE, MFS, MTT, MWW, NES, TON, SFG, STF, STI, TUN, PUN, or a combination thereof.

Particular examples of suitable oligomerization catalysts can include ZSM-5, ZSM-11, ZSM-12, ZSM-22, ZSM-23, ZSM-35, ZSM-48, and combination thereof. Particularly useful catalysts may be selected from the group of zeolites having an MRE-type IZA framework, e.g., ZSM-48 catalyst.

Exemplary silicoaluminophosphates that may be useful herein include one or a combination of SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, and SAPO-56.

The methanol conversion catalyst and the oligomerization catalyst may be the same or different. In particular embodiments, the methanol conversion and oligomerization catalyst are selected from the group of zeolites having an MRE-type IZA framework. In more particular embodiments, the methanol conversion and the oligomerization is accomplished by ZSM-48 catalyst.

Other catalysts that may be useful herein are described in U.S. Pat. Nos. 7,767,611; 7,449,169; 7,198,711; 7,081,556; 6,709,572; 6,673,978; 6,469,226; 6,350,428; 6,221,324; 5,710,085; 5,639,931; 5,536,483; 5,457,078; 5,367,100; 5,296,428; 5,232,579 5,146,029; 4,845,063; 4,872,968; 4,076,842; 4,046,859; 4,035,430; 4,021,331; 4,016,245; 3,972,983; 3,965,205; 3,832,449; 3,709,979; 3,702,886; 3,303,069; and RE 28,341. As well as, U.S. Patent Application Publication Nos. 2006/0194998; 2008/0161619; and 2008/0021253; as well as those published in R. Szostak, Handbook of Molecular Sieves, Van Nostrand Reinhold, New York, N.Y. (1992). Each of these disclosures is incorporated herein by reference in its entirety.

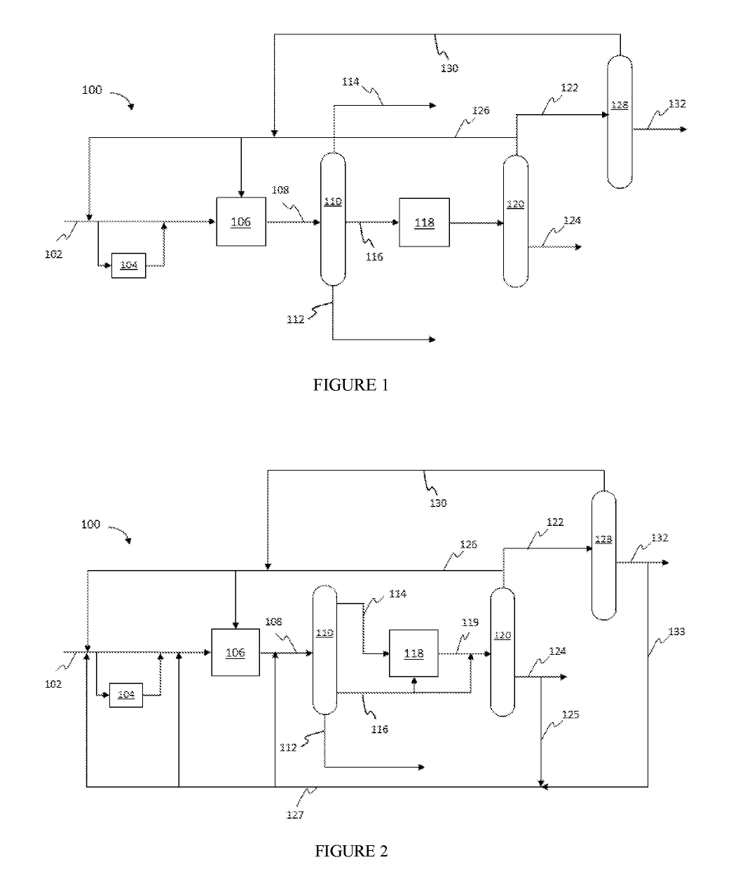

FIG. 1 schematically illustrates a process 100 wherein an oxygenate-containing feed is provided, e.g., via line 102 to methanol conversion reactor 106, having a methanol conversion catalyst therein. Optionally, at least a portion of the feed in line 102 may be passed through dehydration unit 104. The feed may be preheated to a desired reaction temperature (e.g., 330° C. to 370° C.) by means of a heat exchanger or other appropriate hardware (not shown) prior to being provided to the reactor 106. Reactor 106 may be any suitable reactor design, fixed, fluid, or moving bed, particularly a moving bed reactor. The temperature of the feed should account for the heat of reaction, which measurably increases the temperature of the reactor. The WHSV is adjusted to achieve a desired oxygenate conversion. The feed preheat temperature and the feed WHSV may be controlled to maintain the desired conversion.

Optionally, a portion of the feed from line 102 may bypass (not shown) the methanol conversion reactor to be provided to the oligomerization reactor 118, e.g., by combination with the contents of line 114 and or 116. The portion of the feed that bypasses the methanol conversion reactor can be ≧about 10 vol %, e.g., ≧about 20 vol %, ≧about 30 vol %, ≧about 40 vol %, ≧about 45 vol %, ≧about 50 vol %, ≧about 55 vol %, ≧about 60 vol %, ≧about 70 vol %, ≧about 80 vol %, or ≧about 85 vol %, based on the total volume of the feed.

Additionally or alternatively, the portion of the feed that bypasses the methanol conversion reactor can be ≦about 90 vol %, e.g., ≦about 85 vol %, ≦about 80 vol %, ≦about 70 vol %, ≦about 60 vol %, ≦about 55 vol %, ≦about 50 vol %, ≦about 45 vol %, ≦about 40 vol %, ≦about 30 vol %, or ≦about 20 vol %. Ranges of the amount of the feed that bypasses the methanol conversion reactor expressly disclosed include all combinations of any of the above-enumerated values; e.g., 10 to 90 vol %, 20 to 80 vol %, 30 to 70 vol %, 40 to 60 vol %, 45 to 55 vol %, etc.

The methanol conversion catalyst in reactor 106 can convert at least a portion of the oxygenate in the feed to an intermediate composition that comprises olefins and or aromatics. In particular embodiments, the methanol conversion reactor can provide an effluent stream 108 comprising ≧about 10 wt % aromatics, e.g., ≧about 15 wt %, ≧about 20 wt %, ≧about 25 wt %, ≧about 30 wt %, ≧about 35 wt %, or ≧about 40 wt % aromatics, based on the total weight of the effluent of the reactor 106. Additionally or alternatively, the methanol conversion reactor can provide an effluent stream 108 comprising ≦about 45 wt % aromatics, e.g., ≦about 40 wt %, ≦about 35 wt %, ≦about 30 wt %, ≦about 25 wt %, ≦about 20 wt %, or ≦about 15 wt % aromatics, based on the total weight of the effluent of the reactor 106. Ranges of aromatics content of the effluent form the methanol conversion reactor expressly disclosed include all combinations of any of the above-enumerated values; e.g., about 10 wt % to about 45 wt %, about 15 wt % to about 40 wt %, about 20 wt % to about 35 wt %, about 25 wt % to about 30 wt %, etc. In particular embodiments, the effluent can comprise about 12 wt % to about 19 wt % aromatics, where the feed is exposed to a ZSM-48 catalyst at a pressure between about 15 psig and about 90 psig and at a temperature above 450° C. Generally, lower aromatics content can be desirable, because a lower selectivity for aromatics can allow higher yields of olefin from this step of the process.

The effluent of reactor 106 including olefins in the intermediate composition may be directed, e.g., via line 108, to a first separation unit 110. Separation unit 110 may be any type of separation unit suitable for separating an olefin-containing stream from the effluent of the methanol conversion reactor. In certain embodiments, the first separation unit can comprise a 3-phase settler and/or a water knockout drum. In other embodiments, separation unit 110 may comprise or be a membrane. Advantageously, in some embodiments the separation unit is not a distillation column, thereby making the process less capital-intensive. In some embodiments, the first separation unit 110 may advantageously be operated to remove only a portion of the water from reactor 108 effluent. Thus, the gas stream 114 may include ≦about 15 wt % water, e.g., ≦about 12 wt %, ≦about 10 wt %≦about 8.0 wt %, ≦about 6.0 wt %, ≦about 4.0 wt %, ≦about 2.0 wt %, ≦about 1.0 wt %, ≦about 0.5 wt %, ≦about 0.2 wt % water, ≦about 500 wppm water. Additionally or alternatively, the olefin-containing gas stream 114 may include ≧about 0 wt % water, e.g., ≧about 500 wppm, ≧about 0.2 wt %, ≧about 0.5 wt %, ≧about 1.0 wt %, ≧about 2.0 wt %, ≧about 4.0 wt. %, ≧about 6.0 wt %, ≧about 8.0 wt %, ≧about 10 wt %, or ≧about 12 wt % water. Ranges of the amount of water in olefin-containing gas stream 114 expressly disclosed can include all combinations of any of the above-enumerated values, e.g., about 0 wt % to about 15 wt % water, about 500 wppm to about 12 wt % water, about 0.2 wt % to about 10 wt % water, about 0.5 wt % to about 8.0 wt % water, about 1.0 wt % to about 6.0 wt % water, about 2.0 to about 4.0 wt % water, about 500 wppm to about 2.0 wt % water, etc.

By-product water may be removed from the system, e.g., via line 112. The first separation unit may additionally or alternately separate an olefin-containing gas stream 114 and an C₃⁻ liquid stream 116 from the effluent in line 108.

At least a portion of the olefin-containing stream 114 may be provided to oligomerization reactor 118, where it can be contacted with the oligomerization catalyst. Reactor 108 may be any suitable reactor design, fixed, fluid, or moving bed, particularly a moving bed reactor. In certain embodiments, the oligomerization reactor 118 can be a tubular reactor, e.g., comprising multiple straight tubes, such as between 1 and 3 inches in diameter packed into a cylindrical shell between two tube sheets, such as described in U.S. Pat. No. 7,803,332, the disclosure of which is hereby incorporated in its entirety by reference.

Oligomerization reactor 118 may additionally or alternatively convert at least a portion of the olefin-containing C₃⁻ liquid stream 116 to an effluent mixture 119 comprising gasoline boiling range and distillate boiling range components. In particular embodiments, gasoline boiling range products in C₃⁺ liquid stream 116 can be provided to oligomerization reactor 118 for conversion to distillate boiling range products. Additionally or alternatively, gasoline and/or distillate boiling range products in C₃⁺ liquid stream 116 may be sent to a second separation unit 120 for recovery. Optionally, effluent mixture 119 may be provided to the second separation unit 120, e.g., a distillation column, operable to separate primarily C₉⁻ gasoline-boiling range component, optionally having olefins therein, e.g. via line 122, and C₁₀⁺ distillate boiling range components 124. At least a portion of the gasoline-boiling range components 122 may be recycled, e.g., via line 126, to be contacted with the feed and/or to the methanol conversion reactor 106. Any un-recycled portion remaining in line 122 may be directed to a third separation unit 128, e.g., a still or distillation column, operable to separate the relatively small amounts of C₃⁻ as an overhead stream 130 from the C₄⁺ gasoline components exiting the third separation unit 118 via line 132. As is known in the art, the C₄⁺ gasoline components in line 132 can be fractionated between 1,2,4 trimethylbenzene and durene in order to control the durene content of the resulting gasoline.

An additional benefit of the gasoline and/or distillate boiling range products are that such products are substantially free of or completely free of sulfur. Current refined gasoline produced from petroleum contains sulfur. Significant and expensive hydroprocessing is required to reduce sulfur to regulatory standards. This current process results in a refined hydrocarbon that is substantially free of or completely free of sulfur without the need to perform such hydroprocessing.

FIG. 2 schematically illustrates a process 200, wherein an oxygenate-containing feed is provided via line 202 to optional dehydration unit 204 or to a combined methanol conversion/oligomerization reactor 206. Reactor 206 may operate as a dual catalyst reactor, e.g., a methanol conversion catalyst and an oligomerization catalyst. In particular embodiments, a single catalyst, e.g., ZSM-48, can provide both functions. Reactor 206 may be of any suitable type, e.g., fixed, fluid, or moving bed. Reactor 206 can be operated under a first set of conditions where methanol conversion can advantageously be favored. After a desired time, reactor 206 can be operated under a second set of conditions where oligomerization can advantageously be favored. Any conditions consistent with those described herein above may be used. Alternatively, where reactor 206 is a fixed or moving bed reactor, a temperature gradient across the bed may be used. The gradient should be established such that the methanol conversion can be initially preferable.

The reactor 206 can produce an effluent mixture comprising water, gasoline boiling range components, and distillate boiling range components. Optionally, the effluent mixture may be cooled by any convenient means (not shown). The effluent mixture produced by reactor 206 may be conducted via conduit 208 for separation into any desirable fractions in a first separation unit 210. For example, the effluent in conduit 208 may be separated to remove water (e.g. as described and to the extent, described for process 100) from the portion of effluent 208 that is recycled via conduit 214 for further reaction in the oligomerization reactor 206. Distillate-containing product fraction can exit the first separator via, e.g., line 216 for further purification. For example, distillate-containing product fraction in conduit 216 may be directed to a second separator 220 operable to separate primarily C₉⁻ gasoline-boiling range component, optionally having olefins therein, e.g. via line 222, and C₁₀⁺ distillate boiling range components 224. At least a portion of the gasoline-boiling range components 222 may be recycled, e.g., via line 226, to be contacted with the feed and/or to the methanol conversion reactor 106. Likewise, at least a portion of C₁₀⁺ distillate boiling range components 224 may also be recycled via line 227 to, e.g., feed line 202 via conduit 228 and/or 229 and or to the reactor 206 via, e.g., line 230. Any un-recycled portion remaining in line 222 may be directed to a third separation unit 232, e.g., a still or distillation column, operable to separate the relatively small amounts of C₃⁻ as an overhead stream 234 from the C₄⁺ gasoline components exiting the third separation unit 232. Overhead stream 234, typically although not necessarily, can be recycled to, e.g., feed line 202 via conduit 235 and/or 237 and or to the reactor 206 via, e.g., line 230. As is known in the art, the C₄⁺ gasoline components in line 236 can be fractionated between 1,2,4 trimethylbenzene and durene in order to control the durene content of the resulting gasoline. Additionally or alternatively, at least a portion of the C₄⁺ gasoline components in line 236 may be recycled via, e.g., conduit 238 to, e.g., feed line 202 via conduit 228 and/or 229 and/or to the reactor 206 via, e.g., line 230.

One advantage of particular embodiments can include the ability of the process to provide a desirable ratio of products. Thus, the (weight) ratio of gasoline boiling range components to distillate boiling range components (G:D ratio) may be ≦about 1.0, e.g., ≦about 0.90, ≦about 0.80, ≦about 0.75, ≦about 0.70, ≦about 0.65, ≦about 0.60, ≦about 0.55, ≦about 0.50, ≦about 0.45, ≦about 0.40, ≦about 0.35, or ≦about 0.30, on a dry basis. Additionally or alternatively, the G:D (weight) ratio may be ≧about 0.25, e.g., ≧about 0.30, ≧about 0.35, ≧about 0.40, ≧about 0.45, ≧about 0.55, ≧about 0.60, ≧about 0.65, ≧about 0.70, ≧about 0.75, ≧about 0.80, ≧about 0.85, or ≧about 0.90. Ranges of the G:D ratio of the effluent mixture expressly disclosed include all combinations of any of the above-enumerated values; e.g., about 0.25 to about 1.0, about 0.30 to about 0.90, about 0.35 to about 0.85, about 0.40 to about 0.80, about 0.45 to about 0.75, about 0.50 to about 0.70, about 0.55 to about 0.65, about 0.40 to about 0.55, about 0.40 to about 0.50, and the like. In some particular embodiments, e.g., single reactor process 200, the process can provide about 30 wt % gasoline boiling range products, about 65 wt % distillate boiling range products, and about 5 wt % lights gases, on a dry basis.

### Additional or Alternative Embodiments

Embodiment 1. A process for forming a refined hydrocarbon comprising: (a) providing a first mixture comprising ≧10 wt % of at least one oxygenate, based on the weight of the first mixture; (b) contacting at least a portion of the feed with a methanol conversion catalyst under suitable conditions including a first pressure, P₁, to yield an intermediate composition including olefins having at least two carbon atoms; (c) introducing at least a portion of the intermediate composition to an oligomerization catalyst under suitable conditions including a second pressure, P₂, to yield an effluent mixture comprising gasoline boiling range components and distillate boiling range components, wherein the P₂=P₁±200 psi, particularly ±175 psi, ±150 psi, ±125 psi, ±100 psi, ±75 psi, ±50 psi, ±40 psi, ±30 psi, ±25 psi, ±20 psi, ±15 psi, ±10 psi, ±5 psi, or ±2.5 psi; and (d) recovering the gasoline boiling range components and distillate boiling range components.

Embodiment 2. A system for forming a refined hydrocarbon comprising: (a) a feed comprising ≧10 wt % of at least one oxygenate, based on the weight of the first mixture; (b) a first reaction vessel including a first reaction stage containing a methanol conversion catalyst in fluid communication with at least a portion of the feed for contact with the methanol conversion catalyst maintained under suitable conditions including a first pressure, P₁, to yield an intermediate composition including olefins having at least two carbon atoms; (c) a second reaction vessel and/or a second reaction stage containing an oligomerization catalyst in fluid communication with at least a portion of the intermediate composition, the second reaction vessel maintained under suitable conditions including a second pressure, P₂, to yield and effluent mixture comprising gasoline boiling range components and distillate boiling range components; and (d) a recovery system in fluid communication with the second reaction vessel to separate the gasoline boiling range components and distillate boiling range components from the effluent mixture, wherein P₂=P₁±200 psi, particularly ±175 psi, ±150 psi, ±125 psi, ±100 psi, ±75 psi, ±50 psi, ±40 psi, ±30 psi, ±25 psi, ±20 psi, ±15 psi, ±10 psi, ±5 psi, or ±2.5 psi.

Embodiment 3. The system or process of Embodiment 1 or 2, wherein the oxygenate comprises methanol, dimethyl ether, or a mixture thereof.

Embodiment 4. The system or process of any of Embodiments 1-3, wherein the process is essentially free of a compression step between steps (b) and (c).

Embodiment 5. The system or process of any of Embodiments 1-4, wherein the intermediate composition comprises ≧about 40 wt %, particularly, ≧about 45 wt %, ≧about 50 wt %, ≧about 55 wt %, ≧about 60 wt %, ≧about 65 wt %, ≧about 70 wt %, ≧about 75 wt %, ≧about 80 wt %, ≧about 85 wt %, ≧about 90 wt %, ≧about 95 wt %, or ≧about 99 wt % olefins.

Embodiment 6. The system or process of any of Embodiments 1-5, where in the effluent mixture comprises ≧about 50 wt %, particularly ≧about 55 wt %, ≧about 60 wt %, ≧about 65 wt %, ≧about 70 wt %, ≧about 75 wt %, ≧about 80 wt %, ≧about 85 wt %, ≧about 90 wt %, ≧about 95 wt %, or ≧about 99 wt % distillate boiling range components.

Embodiment 7. The system or process of any of Embodiments 1-6, wherein the methanol conversion catalyst is selected from aluminosilicate zeolites having a microporous surface area ≧150 m²/g, 160 m²/g, 165 m²/g, ≧200 m²/g, ≧250 m²/g, ≧300 m²/g, ≧350 m²/g, ≧400 m²/g, ≧450 m²/g, ≧500 m²/g, ≧550 m²/g, ≧600 m²/g, ≧650 m²/g, ≧700 m²/g, ≧750 m²/g, ≧800 m²/g, ≧850 m²/g, ≧900 m²/g, ≧950 m²/g, or ≧1000 m²/g.

Embodiment 8. The system or process of any of Embodiments 1-7, wherein the methanol conversion catalyst has a molar ratio of silicon to aluminum from 10 to 100, for example from 30 to 50 or from 42 to 48.

Embodiment 9. The system or process of any Embodiments 1-8, wherein methanol conversion catalyst has a hexane cracking activity ≧20, e.g., of about 130.

Embodiment 10. The system or process of any of Embodiments 1-9, wherein the methanol conversion catalyst has an IZA framework type selected from the group consisting of BEA, EUO, FER, IMF, LAU, MEL, MFI, MRE, MFS, MTT, MWW, NES, TON, SFG, STF, STI, TUN, PUN, and combinations thereof, for instance MRE, such as wherein the methanol conversion catalyst comprises or is a ZSM-48 catalyst.

Embodiment 11. The system or process of any of Embodiments 1-10, wherein the oligomerization catalyst has an IZA framework type selected from the group consisting of BEA, EUO, FER, IMF, LAU, MEL, MFI, MRE, MFS, MTT, MWW, NES, TON, SFG, STF, STI, TUN, PUN, and combinations thereof, for instance MRE, such as wherein the methanol conversion catalyst comprises or is a ZSM-48 catalyst.

Embodiment 12. The process of any of Embodiments 1 and 3-11, wherein contacting at least a portion of the feed with a methanol conversion catalyst occurs in a first reaction vessel and introducing at least a portion of the intermediate composition to an oligomerization catalyst occurs in a second reaction vessel.

Embodiment 13. The process of any of Embodiments 1 and 3-12, further comprising recycling at least a portion of the separated gasoline boiling range components containing C₄⁺ olefins to the feed to be contacted with the methanol conversion catalyst to yield C₅⁺ branched paraffins and C₇⁺ aromatics.

Embodiment 14. The system of any of Embodiments 2-11, further comprising a recycling system for recycling at least a portion of the separated gasoline boiling range components containing C₄⁺ olefins to the feed to be contacted with the methanol conversion catalyst to yield C₅⁻ branched paraffins and C₇⁺ aromatics.

Embodiment 15. The process of Embodiment 13, wherein the portion of the separated gasoline boiling range components comprises from about 40 wt % to about 90 wt % of the total feed to the methanol conversion catalyst.

Embodiment 16. The system of Embodiment 14, wherein the portion provided by the recycling system comprises from about 40 wt % to about 90 wt % of the total feed to the methanol conversion catalyst.

Embodiment 17. The system or process of any of Embodiments 1-16, wherein the methanol conversion catalyst converts from about 90% to about 95% of the oxygenate in the feed.

Embodiment 18. The process of any of Embodiments 1, 3-13, 15, and 17, further comprising separating C2″ gas and water from the intermediate composition, for example in a three phase settler apparatus.

Embodiment 19. The system of any of Embodiments 2-11, 14, and 16-17, further comprising a separation unit for separating C₂⁻ gas and water from the intermediate composition, such as a three phase settler apparatus.

Embodiment 20. The process of any of Embodiments 1, 3-13, 15, and 17-18, wherein separating the gasoline boiling range components and distillate boiling range components includes fractionating the gasoline boiling range components and distillate boiling range components in at least one distillation column.

Embodiment 21. The system of any of Embodiments 2-11, 14, 16-17, and 19, wherein separating the gasoline boiling range components and distillate boiling range components includes fractionating the gasoline boiling range components and distillate boiling range components in at least one distillation column.

Embodiment 22. The system or process of Embodiment 21 or 22, comprising a first distillation column for separating a C₁₀⁺ distillate boiling range component and a C₉⁻ overhead component, and a second distillation column for receiving the C₉⁻ overhead component from the first distillation column and separating a C₃⁻ overhead component and C₄⁺ gasoline boiling range component.

Embodiment 23. The system or process of any of Embodiments 1-22, wherein the methanol conversion catalyst is maintained in a first vessel, such as a fixed bed adiabatic reactor, maintained at a temperature of about 330° C. to about 550° C., e.g., of about 485° C., and at a pressure of about 50 psig to about 125 psig, e.g., from about 75 psig to about 100 psig or from about 85 psig to about 95 psig.

Embodiment 24. The system or process of any of Embodiments 1-23, wherein the oligomerization catalyst is maintained in a second vessel, such as a tubular reactor, maintained at a temperature of about 100° C. to about 300° C., of about 150° C. to about 250° C., of about 175° C. to about 225° C., or at about 200° C., and at a pressure of about 50 psig to about 125 psig, e.g., from about 75 psig to about 100 psig or from about 85 psig to about 95 psig.

Embodiment 25. A hydrocarbon product of the system or process of any of Embodiments 1-24.

Embodiment 26. The hydrocarbon product of embodiment 25, wherein the product of the system or process is substantially sulfur free.

### EXAMPLES

An example of the performance of the preferred H-ZSM-48 catalyst is shown in FIG. 1. The H-ZSM-48 catalyst used in this example has silicon to aluminum ratio of 45, a microporous surface area of 162 g/m², and a hexane cracking activity of 130. Methanol is contacted with the catalyst at 485° C., and 90 psig at a WHSV of 2 hr⁻¹. The olefin yield is 37.4 wt % of the carbon-containing products. The most abundant olefin product from the conversion of methanol on H-ZSM-48 is propene, accounting for 37.5 wt % of the total olefins. The reactor temperature is lower and propene is contacted with H-ZSM-48 at 200° C. and 90 psig at a WHSV of 2 hr⁻¹. The distillate fraction yield (boiling between 330° F.-730° F.) is 65 wt. % of the product. Table 1 reports the distribution of carbon containing products for the conversion of methanol on H-ZSM-48. Table 2 reports the product distribution for the conversion of propene on H-ZSM-48.

TABLE 1
Yield (wt. %)

| | |
|---|---|
| Methanol | 14.2 |
| DME | 9.4 |
| Methane | 5.0 |
| Total Olefins | 37.4 |
| Ethene | 2.3 |
| Propene | 13.4 |
| Butenes | 12.6 |
| C₅⁺ olefins | 9.1 |
| Paraffins | 9.5 |
| Aromatics | 0.0 |
| CO | 16.1 |
| CO₂ | 0.4 |

TABLE 2

| Product b.p. (° F.) | Yield (wt %) |
|---|---|
| <330 | 17 |
| 330-730 (distillate) | 65 |
| >730 | 18 |

In another set of studies, the oligomerization of propene at 200° C. and 1.66 WHSV is compared at different pressures for H-ZSM-48 and H-ZSM-5. As shown in Table s3 and 4, when the oligomerization reactor is conducted in the presence of H-ZSM-5 at pressures above 200 psig, about 80% distillate boiling range products are produced. At a lower pressure (e.g. 90 psig), however, H-ZSM-5 produces only about 44% distillate. In comparison, at 90 psig, H-ZSM-48 makes 57% distillate.

TABLE 3
Yields for the conversion of propene on H-ZSM-48 at
~200° C. and ~1.66 WHSV

| | Yield (wt %) | | |
|---|---|---|---|
| Product b.p. (° F.) | 90 psig | 200 psig | 800 psig |
| <~330 | 16 | 13 | 13 |
| ~330 to ~730 (distillate) | 57 | 75 | 73 |
| >~730 | 12 | 13 | 14 |

TABLE 4
Yields for the conversion of propene on
H-ZSM-5 at ~200° C. and ~1.66 WHSV

| | Yield (wt %) | | |
|---|---|---|---|
| Product b.p. (° F.) | ~90 psig | ~200 psig | ~800 psig |
| C5 to ~330 | 29 | 13 | 15 |
| ~330 to ~730 (distillate) | 46 | 75 | 79 |
| >~730 | 8 | 8 | 6 |

In yet another set of studies, the effect of water on propene oligomerization in the presence of ZSM-48 at varying temperatures at a pressure of ˜800 psig and a WHSV of about 1.7 is compared.

TABLE 5
Yields for the conversion of propene on H-ZSM-48
at ~800 psig and ~1.7 hr⁻¹

| Water wt % | Reactor | | ~330 to ~730° F. | |
|---|---|---|---|---|
| in Propene | Temp (° F.) | <~330° F. | (distillate) | ~730+° F. |
| ~0% | ~392 | ~31 | ~65 | ~4 |
| ~10% | ~392 | ~99 | ~1 | ~0 |
| ~5% | ~392 | ~65 | ~35 | ~1 |
| ~15% | ~392 | ~95 | ~5 | ~0 |
| ~0% | ~437 | ~17 | ~67 | ~17 |
| ~10% | ~437 | ~68 | ~30 | ~2 |
| ~10% | ~482 | ~63 | ~34 | ~3 |
| ~10% | ~527 | ~52 | ~46 | ~2 |

As Table 5 shows, the oligomerization reaction can still produce distillate at acceptable yield even when the feed includes water at a concentration of <about 15 wt %.

All documents described herein are incorporated by reference herein for purposes of all jurisdictions where such practice is allowed, including any priority documents and/or testing procedures to the extent they are not inconsistent with this text, provided however that any priority document not named in the initially filed application or filing documents is NOT incorporated by reference herein. As is apparent from the foregoing general description and the specific aspects, while forms of the invention have been illustrated and described, various modifications can be made without departing from the spirit and scope of the invention. Accordingly, it is not intended that the invention be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa. Aspects of the invention include those that are substantially free of or essentially free of any element, step, composition, ingredient or other claim element not expressly recited or described.

1. A process for forming a refined hydrocarbon comprising:
(a) providing a first mixture comprising ≧10 wt % of at least one oxygenate, based on the weight of the first mixture;
(b) contacting at least a portion of the first mixture with a methanol conversion catalyst under suitable conditions including a first pressure, P₁, to yield an intermediate composition including olefins having at least two carbon atoms;
(c) introducing at least a portion of the intermediate composition to an oligomerization catalyst under suitable conditions including a second pressure, P₂, to yield an effluent mixture comprising gasoline boiling range components and distillate boiling range components, wherein the P₂=P₁±200 psi; and
(d) recovering at least a portion of the gasoline boiling range components and distillate boiling range components.

2. The process of claim 1, wherein the oxygenate comprises methanol, dimethyl ether, or a mixture thereof.

3. The process of claim 1, wherein the process is essentially free of a compression step between steps (b) and (c).

4. The process of claim 1, wherein the methanol conversion catalyst is selected from aluminosilicate zeolites having a microporous surface area≧150 m²/g.

5. The process of claim 1, wherein the methanol conversion catalyst has a molar ratio of silicon to aluminum of 10 to 100.

6. The process of claim 1, wherein the methanol conversion catalyst has an IZA framework type selected from the group consisting of BEA, EUO, FER, IMF, LAU, MEL, MFI, MRE, MFS, MTT, MWW, NES, TON, SFG, STF, STI, TUN, PUN, and combinations thereof.

7. The process of claim 1, wherein the methanol conversion catalyst is selected from the group of zeolites having an MRE framework type.

8. The process of claim 1, wherein the methanol conversion catalyst comprises a ZSM-48 catalyst.

9. The process of claim 1, wherein the oligomerization catalyst has an IZA framework type selected from the group consisting of BEA, EUO, FER, IMF, LAU, MEL, MFI, MRE, MFS, MTT, MWW, NES, TON, SFG, STF, STI, TUN, PUN, and combinations thereof.

10. The process of claim 1, wherein the oligomerization catalyst is selected from the group of zeolites having an MRE framework type.

11. The process of claim 1, wherein the oligomerization catalyst comprises ZSM-48.

12. The process of claim 1, wherein the methanol conversion catalyst and the oligomerization catalyst each comprise H-ZSM-48.

13. The process of claim 1, wherein the methanol conversion catalyst is maintained in a first vessel maintained at a temperature of about 330° C. to about 550° C. and a pressure of about 50 psig to about 125 psig.

14. The process of claim 1, wherein the oligomerization catalyst is maintained in a second vessel maintained at a temperature of about 100° C. to about 300° C. and a pressure of about 50 psig to about 125 psig.

15. A system for forming a refined hydrocarbon comprising:
(a) a feed comprising ≧10 wt % of at least one oxygenate, based on the weight of the first mixture;
(b) a first reaction vessel containing a methanol conversion catalyst in fluid communication with at least a portion of the feed for contact with the methanol conversion catalyst maintained under suitable conditions including a first pressure, P₁, to yield an intermediate composition including olefins having at least two carbon atoms;
(c) a second reaction vessel containing an oligomerization catalyst in fluid communication with at least a portion of the intermediate composition, the second reaction vessel maintained under suitable conditions including a second pressure, P₂, to yield and effluent mixture comprising gasoline boiling range components and distillate boiling range components; and
(d) a recovery system in fluid communication with the second reaction vessel to separate at least a portion of the gasoline boiling range components and distillate boiling range components from the effluent mixture,
wherein the P₂=P₁±200 psi.

16. The system of claim 15, wherein the oxygenate comprises methanol, dimethyl ether, or a mixture thereof.

17. The system of claim 15, wherein the process is essentially free of a compression step between steps (b) and (c).

18. The system of claim 15, wherein the methanol conversion catalyst is selected from aluminosilicate zeolites having a microporous surface area≧150 m²/g.

19. The system of claim 15, wherein the methanol conversion catalyst has a molar ratio of silicon to aluminum of 10 to 100.

20. The system of claim 15, wherein the methanol conversion catalyst has an IZA framework type selected from the group consisting of BEA, EUO, FER, IMF, LAU, MEL, MFI, MRE, MFS, MTT, MWW, NES, TON, SFG, STF, STI, TUN, PUN, and combinations thereof.

21. The system of claim 15, wherein the methanol conversion catalyst is selected from the group of zeolites having an MRE-type IZA framework.

22. The system of claim 15, wherein the methanol conversion catalyst comprises a ZSM-48 catalyst.

23. The system of claim 15, wherein the oligomerization catalyst has an IZA framework type selected from the group consisting of BEA, EUO, FER, IMF, LAU, MEL, MFI, MRE, MFS, MTT, MWW, NES, TON, SFG, STF, STI, TUN, PUN, and combinations thereof.

24. The system of claim 15, wherein the oligomerization catalyst is selected from the group of zeolites having an MRE-type IZA framework.

25. The system of claim 15, wherein the oligomerization catalyst is a ZSM-48 catalyst.

26. The system of claim 15, wherein the methanol conversion catalyst and the oligomerization catalyst comprise ZSM-48

27. The system of claim 15, wherein the methanol conversion catalyst is maintained in the first vessel maintained at a temperature of about 330° C. to about 550° C. and a pressure of about 50 psig to about 125 psig.

28. The process of claim 15, wherein the first vessel is a fixed bed adiabatic reactor.

29. The system of claim 15, wherein the oligomerization catalyst is maintained in a second vessel maintained at a temperature of about 100° C. to about 300° C. and a pressure of from about 50 psig to about 125 psig.

30. A system for forming a refined hydrocarbon comprising:
(a) a feed comprising ≧10 wt % of at least one oxygenate, based on the weight of the first mixture;
(b) a first reaction vessel containing a methanol conversion catalyst in fluid communication with at least a portion of the feed for contact with the methanol conversion catalyst maintained under suitable conditions including a first pressure, P₁, to yield an intermediate composition including olefins having at least two carbon atoms, thereafter maintained under a second set of conditions including second pressure P₂, to yield and effluent mixture comprising gasoline boiling range components and distillate boiling range components, wherein the P₂=P₁±200 psi; and
(c) a recovery system in fluid communication with the second reaction vessel to separate at least a portion of the gasoline boiling range components and distillate boiling range components from the effluent mixture.

31. The system of claim 30, wherein the oxygenate comprises methanol, dimethyl ether, or a mixture thereof.

32. The system of claim 30, wherein the methanol conversion catalyst is selected from aluminosilicate zeolites having a microporous surface area≧150 m²/g.

33. The system of claim 30, wherein the methanol conversion catalyst has a molar ratio of silicon to aluminum of 10 to 100.

34. The system of claim 30, wherein the methanol conversion catalyst has an IZA framework type selected from the group consisting of BEA, EUO, FER, IMF, LAU, MEL, MFI, MRE, MFS, MTT, MWW, NES, TON, SFG, STF, STI, TUN, PUN, and combinations thereof.

35. The system of claim 30, wherein the methanol conversion catalyst is selected from the group of zeolites having an MRE-type IZA framework.

36. The system of claim 30, wherein the methanol conversion catalyst comprises a ZSM-48 catalyst.

37. The system of claim 30, wherein the methanol conversion catalyst is maintained in the first vessel maintained at a temperature of about 330° C. to about 550° C. and a pressure of about 50 psig to about 125 psig.

38. The system of claim 30, wherein the first vessel is a fixed bed adiabatic reactor.

39. The system of claim 30, wherein the second set of suitable conditions include a temperature of about 100° C. to about 300° C. and a pressure of about 50 psig to about 125 psig.

40. A refined hydrocarbon made by the process of claim 1.

41. The refined hydrocarbon of claim 40, wherein the refined hydrocarbon is substantially free of sulfur.
